(19) United States
(12) Patent Application Publication
(10) Pub. No.: US 2024/0316263 A1
(43) Pub. Date: Sep. 26, 2024

(54) SKIN PATCH DRUG INFUSION DEVICE AND DRUG FILLING METHOD THEREOF

(71) Applicant: MEDTRUM TECHNOLOGIES INC., Shanghai (CN)

(72) Inventor: Cuijun Yang, Shanghai (CN)

(73) Assignee: MEDTRUM TECHNOLOGIES INC., Shanghai (CN)

(21) Appl. No.: 18/269,958

(22) PCT Filed: Aug. 23, 2021
(86) PCT No.: PCT/CN2021/114029

§ 371 Date: Jun. 28, 2023

(30) Foreign Application Priority Data
Jan. 5, 2021 (WO) PCT/CN2021/070207

(51) Int. Cl. A61M 5/142; A61M 5/172

(52) U.S. Cl. CPC A61M 5/14248; A61M 5/1723; A61M 2005/14252

(57) ABSTRACT

A skin patch drug infusion system includes an infusion mechanism module. The infusion mechanism module includes: a reservoir with a drug inlet and a drug outlet for accommodating the drug to be infused; an infusion needle with one end connected to the drug outlet of the reservoir. The other end is inserted subcutaneously implanting drug infusion. The skin patch drug infusion system includes: a control mechanism module, which works collaboratively with the infusion mechanism module to regulate drug infusion; an adhesive patch that attaches the infusion mechanism module and/or the control mechanism module to the skin surface; a filling module. The drug to be infused into the reservoir is filled via the drug inlet, the volume of the filling module is deliberately designed to be greater than the volume of the drug to be infused to ultimately reduce the risk of air being injected into the body.

### TECHNICAL FIELD

The present invention mainly relates to the field of medical instruments, in particular to a skin patch drug infusion system and drug filling method.

### BACKGROUND

The pancreas in a normal person can automatically monitor the amount of glucose in the blood and automatically secrete the required dosage of insulin/glucagon. However, for diabetic patients, the function of the pancreas has been severely compromised, and the pancreas cannot secrete the required dosage of insulin. Therefore, diabetes mellitus is defined as a metabolic disease caused by abnormal pancreatic function, and it is also classified as one of the top three chronic conditions by the WHO. The present medical advancement has not been able to find a cure for diabetes mellitus. Yet, the best the technology could do is control the onset symptoms and complications by stabilizing the blood glucose level for diabetes patients.

Diabetic patients on an Insulin pump need to fill the insulin into the infusion mechanism module and check their blood glucose before infusing insulin into their body. At present, most detection methods can continuously detect blood glucose and send the blood glucose data to the remote device in real-time for the user to view. This detection method is called Continuous Glucose Monitoring (CGM), which requires the detection device to be attached to the surface of the patients' skin, and the sensor carried by the device is inserted into the interstitial fluid for testing. According to the blood glucose (BG) level, the infusion system mimics an artificial pancreas to fill the needs of the required insulin amount via the closed-loop pathway or the semi-closed-loop pathway.

However, it is quite common that some of the infusion mechanism modules will leave the factory with air existed in the fluid pathway and no air aspirating in drug filling process, therefore, increase the risk of air being infused into the body, causing safety hazards.

Therefore, in the prior art, there is an urgent need for a drug infusion system and the filling method that ultimately aspirate the fluid path during the filling process.

### BRIEF SUMMARY OF THE INVENTION

The invention discloses a skin patch drug infusion system. The volume of the filling module is deliberately designed to be greater than the volume of the drug to be infused, to ultimately reduce the risk of air being injected into the body; since the excess space in the filling module could generate a negative pressure that would extract the air in the fluid path. The Invention aims to provide the user with a better experience and lower safety hazards in the meantime.

The Invention discloses a skin patch drug infusion system that includes an infusion mechanism module. The infusion module includes: a reservoir with a drug inlet and a drug outlet for accommodating the drug to be infused; an infusion needle with one end connected to the drug outlet of the reservoir, and the other end is inserted subcutaneously implanting drug infusion; a control mechanism module, which works collaboratively with the infusion mechanism module to regulate drug infusion; an adhesive patch that attaches the infusion mechanism module and/or the control mechanism module to the skin surface; a filling module, where the drug to be infused into the reservoir is filled via the drug inlet. The volume of the filling module is deliberately designed to be greater than the volume of the drug to be infused, and the excess space in the filling module is used to generate negative pressure to aspirate the air in the fluid path.

According to one aspect of the present invention, the volume of the filling module is deliberately designed to be greater than the volume of the reservoir.

According to one aspect of the present invention, the volume of the filling module is 1 mL-2 mL greater than the volume of the reservoir.

According to one aspect of the present invention, the reservoir volume is 1 mL-5 mL.

According to one aspect of the present invention, the reservoir volume is 1 mL-2 mL.

According to one aspect of the present invention, the volume of the filling module is at least 20% greater than the volume of the reservoir.

According to one aspect of the present invention, the drug inlet further includes an elastic seal, which can automatically seal the drug inlet to prevent the drug from leaking after the drug has been filled into the reservoir.

According to one aspect of the present invention, the infusion mechanism module and the control mechanism module are designed separately, and the control mechanism module can be reused.

According to one aspect of the present invention, the infusion mechanism module and the control mechanism module are disposed of in one housing, discarded together after a single-use.

According to one aspect of the present invention, the control mechanism module is provided with multiple first electrical contacts exposed on the surface of the control mechanism module, and the infusion mechanism module is provided with multiple second electrical contacts corresponding to the first electrical contacts, the first electrical contacts and the corresponding second electrical are pressing against each other, thereby electrically connecting the control mechanism module and the infusion mechanism module.

According to one aspect of the present invention, one of the first or second electrical contacts is a rigid metal pin or an elastic conductive member.

According to one aspect of the present invention, it further includes a buzzer, non-closed provided in the control mechanism module.

According to one aspect of the present invention, a flexible circuit board is further provided in the infusion mechanism module.

According to one aspect of the present invention, the infusion mechanism module further includes a case, which includes an upper case and a lower case. The lower case further includes an outward extending portion, and a block is provided outside the outward extending portion.

The invention discloses a drug filling method applied to a skin patch drug infusion system. Before the drug was filled into the reservoir, the excess space in the filling module is used to generate negative pressure to draw out and aspirate the air in the fluid path, which can reduce the risk of air being infused into the body, lower safety hazards, and improve user experience.

The invention discloses a drug filling method applied to a skin patch drug infusion system for filling the drug to be infused into the infusion mechanism module, which comprises: Step 1: Withdraw the drug from the vial into the filling module, where the volume of the filling module is deliberately designed to be greater than the volume of the drug to be infused; Step 2: Insert the filling module into the drug inlet of the infusion mechanism module and get rid of the air in the fluid path of the infusion mechanism module; Step 3: Pull out the filling module and aspirate the air; Step 4: Insert the filling module into the drug inlet of the infusion mechanism module again and fill it into the reservoir of the infusion mechanism module.

According to one aspect of the present invention, it further includes air aspirating between step 1 and step 2.

According to one aspect of the present invention, the air aspirating can be performed in the vial or after the filling module being pulled out from the vial.

According to one aspect of the present invention, the volume of the filling module is deliberately designed to be greater greater than the volume of the reservoir.

According to one aspect of the present invention, the drug filling process can be performed before or after the infusion mechanism module and control mechanism module are electrically connected.

The invention discloses another drug filling method applied to a skin patch drug infusion system. After the drug was filled into the reservoir, the filling module is used to draw out and aspirate the air in the fluid path, reducing the risk of air being infused into the body, lowering safety hazards, and improving user experience.

The invention discloses another drug filling method applied to a skin patch drug infusion system for filling the drug to be infused into the infusion mechanism module, which comprises: Step 1: Withdraw the drug to be infused from a vial into the filling module; Step 2: Insert the filling module into the drug inlet of the infusion mechanism module and fill the drug into the reservoir; Step 3: Pull the push-pull rod of the filling module and draw out the air in the fluid path of the infusion mechanism module.

According to one aspect of the present invention, it further includes air aspirating between step 1 and step 2.

According to one aspect of the present invention, the air aspirating can be performed in the vial or after the filling module being pulled out from the vial.

According to one aspect of the present invention, the drug filling process can be performed before or after the infusion mechanism module and control mechanism module are electrically connected.

Compared with the prior art, the technical solution of the present invention has the following advantages:

In the skin patch drug infusion system disclosed by the present invention, the volume of the filling module is greater than the volume of the drug to be infused. The excess space in the filling module generates negative pressure to draw out the air in the fluid path, which can reduce the risk of air being infused into the body, lower safety hazards, and improve user experience. Secondly, a control mechanism module is provided with multiple first electrical contacts exposed on the surface of the control mechanism module; the infusion mechanism module is provided with multiple second electrical contacts exposed on the surface of the case. The contact area of the electrical contact is small, facilitating the mechanism module design and helps to reduce the volume of the control mechanism module. Thirdly, when the first engaging portions and the second engaging portions are engaged, the first electrical contact and the corresponding second electrical contact press against each other, connecting the control mechanism module and the infusion mechanism module. The control mechanism module and the infusion mechanism module are electrically connected through pressing against each other, which facilitates the optimization of the circuit design and helps improve the electrical connection's reliability.

Since the volume of the filling module is deliberately designed to be greater than the volume of the reservoir, the maximum amount of drug can be filled into the reservoir during the drug filling and air aspirating process, increasing the use time of the drug, reduces the frequency of replacement of the drug infusion system, improves user experience, and improves the accuracy of the actual drug volume filled into the reservoir, thereby improving the accuracy of the drug infusion.

Furthermore, it includes an elastic seal at the drug inlet, automatically sealing the drug inlet to avoid leaking and prevent air entering after the drug has been filled into the reservoir.

In the drug filling method applied to a skin patch drug infusion system disclosed by the present invention, before the drug was filled into the reservoir, the excess space in the filling module allows a negative pressure to be generated, aspirating the air in the fluid path. Therefore, the risk of air being infused into the body, safety hazards have been significantly reduced.

Furthermore, after the drug is inhaled from the vial into the filling module, an air aspirate step is included to reduce the risk of air being filled into the fluid path.

Furthermore, the volume of the filling module is deliberately designed to be greater than the volume of the reservoir, the maximum amount of drug to be infused can be filled into the reservoir during the drug filling and air aspirating process, which increases the use time of the drug, reduces the frequency of replacement of the drug infusion system, improves the user experience, and improves the accuracy of the actual drug volume filled into the reservoir, thereby improving the accuracy of the drug infusion.

Furthermore, the drug filling process can be performed after or before the infusion mechanism module and control mechanism module electrically connected, which improves the versatility and convenience of the operation before the drug infusion.

In another drug filling method applied to a skin patch drug infusion system disclosed by the present invention, after the drug has been filled into the reservoir, the filling module is used to draw out and aspirate the air in the fluid path, which can reduce the risk of air being infused into the body, lower safety hazards, and improve user experience.

Furthermore, after the drug is inhaled from the vial into the filling module, an air aspirate step is also included, which can further reduce the risk of air being filled into the fluid path.

### BRIEF DESCRIPTION OF THE DRAWINGS

FIG. 1a and FIG. 1b are schematic top views of the drug infusion system according to two embodiments of the present invention.

FIG. 2a is a schematic view of the control mechanism module from the front side according to an embodiment of the present invention.

FIG. 2b is a schematic view of the control mechanism module from the bottom side according to an embodiment of the present invention.

FIG. 2c is a schematic view of the control mechanism module without upper housing from the front side according to an embodiment of the present invention.

FIG. 3a is a schematic view of the infusion mechanism module according to an embodiment of the present invention.

FIG. 3b is a side view of the assembly of the control mechanism module and the infusion mechanism module according to an embodiment of the present invention.

FIG. 3c is a schematic top view of the lower case of the infusion mechanism module according to an embodiment of the present invention.

FIG. 3d is a schematic top view of the lower case of the infusion mechanism module according to another embodiment of the present invention.

FIG. 3e is a schematic view of the infusion mechanism module's infusion needle unit according to an embodiment of the present invention.

FIG. 3f is a schematic view of the reservoir and cavity of the infusion mechanism module according to an embodiment of the present invention.

FIG. 4a and FIG. 4b are schematic views of the internal mechanism module of the infusion mechanism module according to an embodiment of the present invention, respectively.

FIG. 5 is a flow chart of a method for the drug filling process according to an embodiment of the present invention.

FIG. 6 is a flow chart of a method for the drug filling process according to another embodiment of the present invention.

### DETAILED DESCRIPTION

As mentioned above, in the prior art, no air was aspirated during the filling process, therefore, increase the risk of air being infused into the body, causing safety hazards.

In order to solve this problem, the present invention provides a drug infusion system. The volume of the filling module is greater than the volume of the drug to be infused. The excess space in the filling module generates negative pressure to draw out the air in the fluid path, reducing the risk of air being infused into the body, mitigating safety hazards, and improving user experience. The present invention also provides a filling method applied to a skin patch drug infusion system. The volume of the filling module is greater than the volume of the drug to be infused. Before the drug was filled into the reservoir, the excess space in the filling module is used to generate negative pressure to draw out and aspirate the air in the fluid path, reducing the risk of air being infused into the body, lower safety hazards, and improve user experience. The present invention also provides another filling method applied to a skin patch drug infusion system. After the drug was filled into the reservoir, the filling module is used to draw out and aspirate the air in the fluid path, reducing the risk of air being infused into the body, lowering safety hazards, and improving user experience.

Various exemplary embodiments of the present invention will now be described in detail regarding the figures. The relative arrangement of the components and the steps, numerical expressions and numerical values outlined in the embodiments are not construed as limiting the scope of the invention.

In addition, it should be understood that, for ease of description, the dimensions of the various components shown in the figures are not necessarily drawn in the actual scale relationship; for example, the thickness, width, length or distance of certain units may be exaggerated relative to other mechanism modules.

The following description of the exemplary embodiments is merely illustrative and does not limit the invention its application or use. The techniques, methods, and devices are known to those of ordinary skill in the art and may not be discussed in detail. However, such techniques, methods, and devices should be considered as part of the specification.

It should be noted that similar reference numerals and letters indicate similar items in the following figures. Therefore, once an item is defined or illustrated in a drawing, it will not be discussed further in the following description of the drawings.

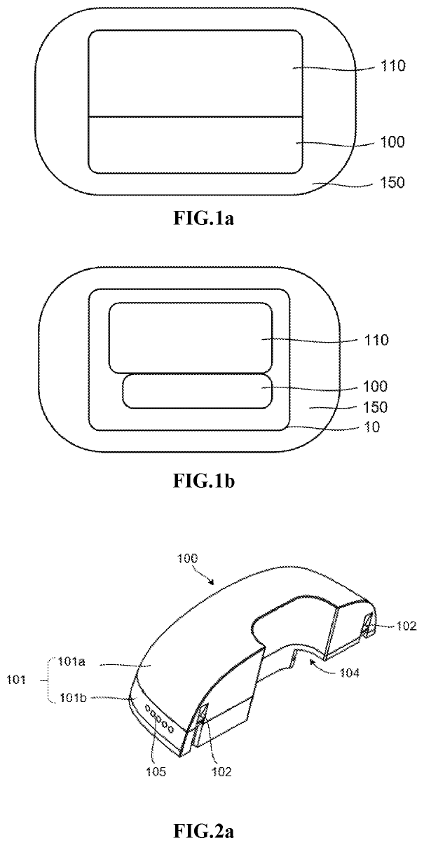

FIG. 1a and FIG. 1b are schematic top views of the drug infusion system according to two embodiments of the present invention.

The skin patch drug infusion system is comprised of an adhesive patch 150, a control mechanism module 100, an infusion mechanism module 110 and a filling module 120.

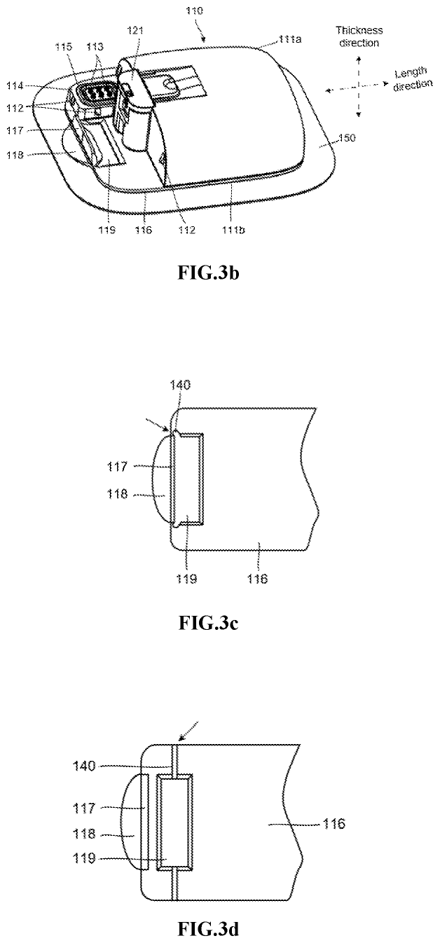

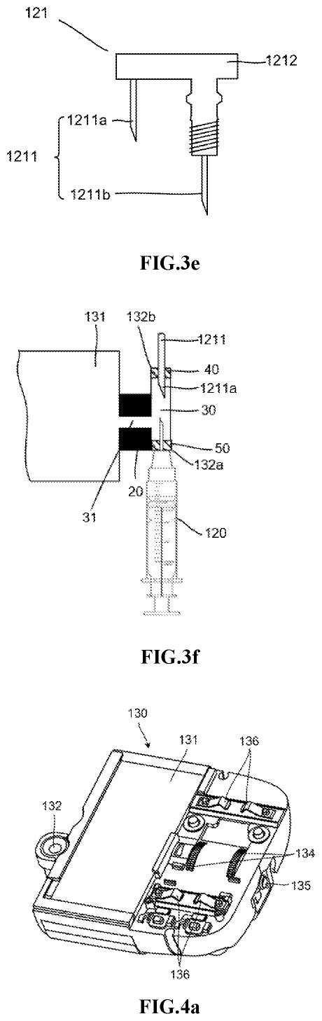

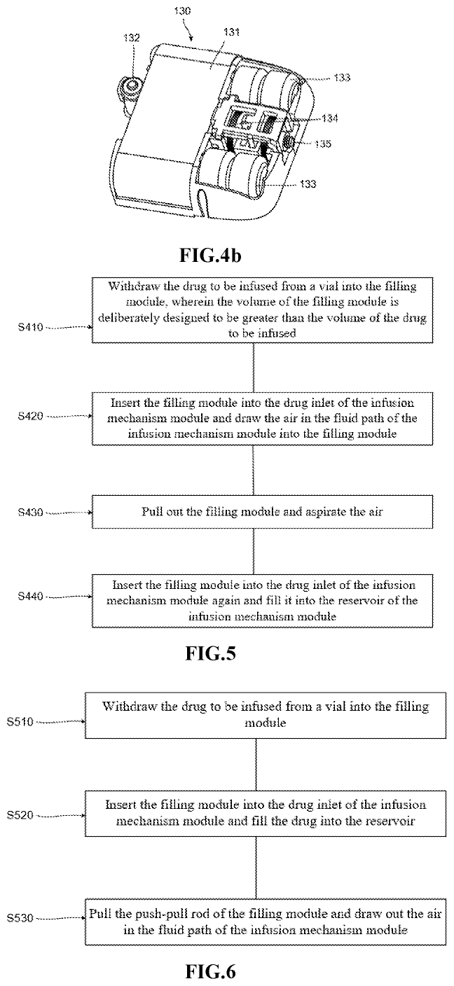

The infusion mechanism module 110 comprises various units for the realization of the mechanical function of drug infusion. Reservoir 131 (as shown in FIG. 4a), which is used to hold the drug to be infused and is internally provided with a piston; Infusion needle unit 121 (as shown in FIG. 3b), with one end of the infusion needle communicating with the drug outlet of the reservoir, and the other end delivered subcutaneously to realize drug infusion; a drive unit (not shown) that moves the piston to make the drug infusion into the body; and other electronic control units and auxiliary units.

The control mechanism module 100 establishes wireless communication with a remote device to receive signals or information from a remote device or a body fluid parameter detection device (such as a continuous blood glucose detection device) and then control the power output of the drive unit of the infusion mechanism module 110 to control drug infusion.

The filling module 120 is used to inhale the drug from the vial into the filling module and fill the drug into reservoir 131 of the infusion mechanism module 110. The drug can be infused into the subcutaneous tissue along the infusion needle by the infusion needle module 121 of the infusion mechanism module 110.

The adhesive patch 150 is used to attach the infusion mechanism module 110 or the control mechanism module 100, or both of them to the skin surface as a whole.

In the embodiment of the present invention, the infusion mechanism module 110 and the control mechanism module 100 are designed separately and connected by a waterproof plug or directly engaged and electrically connected into a whole. Details regarding how the reliability of the electrical connection has been improved when the infusion mechanism module 110 and the control mechanism module 100 are directly engaged and electrically connected into a whole will be described below. The infusion mechanism module 110 can be reused, and the control mechanism module 100 is discarded after a single-use, as shown in FIG. 1a. In another embodiment of the present invention, the infusion mechanism module 110 and the control mechanism module 100 are connected by a wire and disposed of inside the same housing 10. Attached to a certain position of the user's skin by the adhesive patch 150, both units will be discarded together after a single-use, as shown in FIG. 1b.

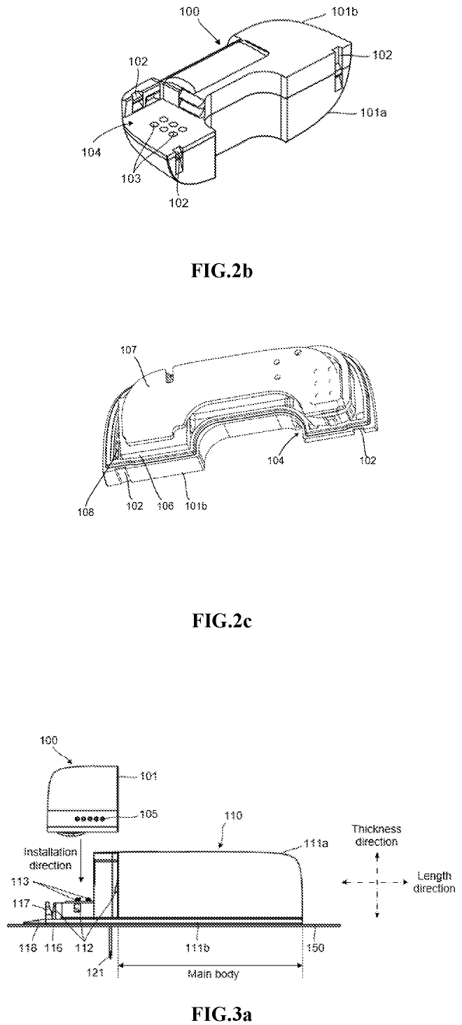

FIG. 2a is a schematic view of the control mechanism module from the front side according to an embodiment of the present invention. FIG. 2b is a schematic view of the control mechanism module from the bottom side according to an embodiment of the present invention. FIG. 2c is a schematic view of the control mechanism module without upper housing 101a from the front side, according to an embodiment of the present invention.

As above mentioned, when the infusion mechanism module 110 and the control mechanism module 100 are directly engaged and electrically connected into a whole, the reliability of the electrical connection can be improved. The two modules and the filling module 120 will be described below, respectively.

Inside the housing 101 of the control mechanism module 100 are disposed of program modules, circuit board(s) 107 and related electronic units for receiving signals or issuing control instructions, as well as other mechanical units or structures necessary for realizing the infusion function, which is not limited herein. The housing 101 includes an upper housing 101a and a lower housing 101b. In another embodiment of the present invention, a power supply 133 can also be provided in the control mechanism module. Preferably, in the embodiment of the present invention, the power supply 133 is provided in the infusion mechanism module 110, which will be described below.

The control mechanism module 100 further includes a first electrical connection 103 exposed on the surface of it. The first electrical connection 103 is used as a circuit connection terminal to connect the internal circuits provided in the control mechanism module 100 and the infusion mechanism module 110, respectively. The embodiment of the present invention does not specifically limit the positions of the first electrical connection 103.

Preferably, in the embodiment of the present invention, the first electrical connection 103 is multiple first electrical contacts 103. Compared with the plug connector used as a connection terminal in the prior arts, the contact area of the electrical contact is much smaller, which provides more flexibility to the mechanism module design, and can effectively reduce the volume of the control mechanism module. At the same time, these smaller electrical contacts can be directly connected to the internal circuit or electrical components. They could also be directly soldered on the circuit board, which helps to optimize the design of the internal circuit and effectively reduce the complexity of the circuit, thereby saving costs and reducing the volume of the infusion system. Furthermore, the electrical contacts are exposed on the surface of the control mechanism module 100 to facilitate electrical connection with connection ends on other mechanism modules.

The type of the first electrical contact 103 includes rigid metal pins or elastic conductive members. Preferably, in the embodiment of the present invention, the first electrical contact 103 is a rigid metal pin. One end of the first electrical contact 103 is electrically connected to the connection end provided inside the control mechanism module 100. In contrast, the other end is exposed on the surface of the lower housing 101b. The rest part of the first electrical contact 103 is tightly embedded in the housing 101, thus keeping the internal control mechanism module 100 isolated from the outside.

The elastic conductive member includes conductive spring, conductive silica gel, conductive rubber, or conductive leaf spring. One end of the elastic conductive member is used to electrically connect with the internal connection end in the control mechanism module 100, while the other end is used to connect with other connection ends electrically.

As in an embodiment of the present invention, the first electrical contact 103 is a conductive spring. When the electrical contacts are in contact with each other, the elasticity of the conductive spring can enhance the reliability of the electrical connection. Similar to the rigid metal pin, one end of the conductive spring is exposed on the surface of the lower housing 101b. In contrast, the remaining part of the conductive spring is tightly embedded in the housing 101 and electrically connected with internal circuits or electrical components. The connection end disposed inside the control mechanism module 100 can be a conductive lead, a specific part of a circuit, or an electrical element.

It should be noted that the “tightly embedded” in the embodiment of the present invention suggests that there is no gap between the electrical contact and the housing 101, keeping the control mechanism module 100 tightly sealed. The following “tightly embedded” has the same meaning as here.

In another embodiment of the present invention, the first electrical contact 103 is a conductive spring, but it is not tightly embedded in the housing 101. Instead, a sealing element is provided in a groove, both of which are disposed around the area where the first electrical contacts 103 are located, thus sealing the electrical contact area and the control mechanism module 100.

In the embodiment of the present invention, the control mechanism module 100 is further provided with the first engaging portions 102, which is used to fasten the second engaging portion 112 disposed on the infusion mechanism module 110 to assemble the control mechanism module 100 infusion mechanism module 110. Details regarding how the mechanism works to enable the electrical connection between the first electrical contacts 103 and the second electrical contacts 113 (as shown in FIG. 2a and FIG. 2b) will be described below.

The first engaging portion 102 and the second engaging portion 112 include one or more hooks, blocks, holes, and slots that can be engaged with each other. The positions of the hooks, blocks, holes, and slots can be flexibly adjusted, according to the shape and mechanism module features of the control mechanism module 100 and the infusion mechanism module 110, such as disposed in the interior or on the surface of the corresponding mechanism module, which is not specifically limited herein.

In the embodiment of the present invention, the control mechanism module 100 is further provided with a concave 104 that fits the convex portion 114 disposed at the bottom of the case of the infusion mechanism module 110, which will be described in detail below. Preferably, the first electrical contacts 103 are provided in the concave 104, as shown in FIG. 2b.

In the embodiment of the present invention, an alarm is non-closed provided in the control mechanism module 100; at least a non-closed area is also provided on the housing 101 of the control mechanism module 100. Whenever the infusion process starts or ends; the infusion system malfunctions; the drug is aspirated; the control mechanism module 100 issues an error command or receives an error message, etc., the alarm issues alarm signals, such as light, sound or vibration, notifying the user to adjust or replace the device in time. The alarm can be one or more lighting lights, audio alarms, and vibration alarms, not specifically limited here.

In the embodiment of the present invention, the non-closed area provided on the housing 101 of the control structure 100 has one or more openings: a circle, a square, a triangle, a polygon, any irregular shape or arbitrary shape. They can be arranged in a single row, multiple rows or any other random arrangement. The non-enclosed area on the housing 101 of the control mechanism module 100 is not limited. Specifically, it may only be provided on the upper housing 101a, the lower housing 101b or the upper housing 101a and lower housing 101b simultaneously. The location of the non-closed area on the upper housing 101a or the lower housing 101b is also not limited. Specifically, it can be provided on the top, front, left or right side of the upper housing 101a or the lower housing 101b. It can also be provided on only one side or provided on multiple sides at the same time. The number, shape, and arrangement of the non-closed area on each side are not limited, as they can be the same or different. That is, the location, shape, size, number, and arrangement of the non-closed area are not limited here, as long as the alarm signal can be sent out. Preferably, in the embodiment of the present invention, the non-enclosed area is an opening provided along the edge of the alarm; that is, the location of the non-enclosed area is adapted to the position of the alarm, which makes it easier for the alarm signal to be sent out and raise the user's attention, reducing the energy consumption of the alarm, optimizing the power consumption configuration of the infusion system and saving production costs.

As the alarm is non-closed provided in the control mechanism module 100, a lower decibel sound emitted from the alarm with a much lower energy consumption would be enough to raise the user's attention, compared to the traditional technical solution in which the alarm is entirely enclosed in the control mechanism module 100. Similarly, the light alarm will be easier to emit from a non-enclosed area than an entirely enclosed area. Especially when the housing 101 of the control mechanism module 100 can only be made of a material with poor light transmission, more energy consumption of the alarm can be reduced as the light will be much easier to emit from the non-enclosed area. When the alarm is a vibration alarm, the weight of the infusion system is reduced due to the existence of the non-enclosed area, and the alarm requires only a small amount of power consumption to generate vibration and be noticed by the user. Based on the reasons mentioned above, when the alarm is a combined form of audio, lighting, and vibration, or an alarm that has multiple alarm signals such as lighting, sound, or vibration, as the alarm is non-closed provided in the control mechanism module 100, it will be easier for the alarm signals to emit from the non-enclosed area and to raise the attention of the user, reducing the energy consumption of the alarm, optimizing the power consumption configuration of the infusion system and saving production costs.

Specifically, in the embodiment of the present invention, the alarm is an audio form. Preferably, in the embodiment of the present invention, the audio alarm is a buzzer 106, amounted on circuit board 107 of the control mechanism module 100. Whenever the infusion process starts or ends; the infusion system malfunctions; the drug is exhausted; the control mechanism module 100 issues an error command or receives an error message, etc., the buzzer 106 will issue alarm signals, such as sound or vibration, notifying the user to adjust or replace the device in time. More specifically, in the embodiment of the present invention, the buzzer 106 is a piezoelectric buzzer.

Preferably, in the embodiment of the present invention, the non-closed area provided on the lower housing 101b of the control mechanism module 100 is a sound-permeable outlet 105 to allow the sound alarm signal from the buzzer 106 to be sent out. As mentioned above, the shape, size, number, arrangement on the housing 101 of the control mechanism module 100 of the sound-permeable outlet 105 are not limited and will not be described here, as long as the sound alarm signal can be emitted. Preferably, in the embodiment of the present invention, the sound-permeable outlet 105 are a row of dense but separated small holes arranged on the side surface of the lower housing 101b. The diameter of the small holes is extremely small, even smaller than the diameter of general raindrops, preventing water drops from entering the control structure 101. The position of the sound-permeable outlet 105 is adapted to the position of the buzzer 106. The overall length span is slightly larger than the edge length of the buzzer 106. The sound alarm signal of the buzzer 106 can be emitted more easily, reducing the energy consumption of the alarm, optimizing the power consumption configuration of the infusion system and saving production costs.

In order to achieve a good sealing effect and ensure the normal operation of the buzzer, a waterproof sound-permeable membrane 108 is provided between the sound-permeable outlet 105 and the buzzer 106. Therefore, the waterproof sound-permeable membrane 108 needs to have a certain porosity to ensure the sound transmission but prevent water molecules penetration. Preferably, in the embodiment of the present invention, the waterproof sound-permeable membrane 108 comprises many small holes with a diameter of 0.1-1 microns, with a waterproof and dust-proof grade equivalent to IP68.

Compared with the traditional technical solution in which the buzzer is entirely enclosed in the control mechanism module 100, a less loud sound signal emitted from the buzzer would be enough to raise the user's attention. The sound-permeable outlet 105 and the waterproof sound-permeable membrane 108 reduce the buzzer's energy consumption, thereby optimizing the power consumption configuration of the infusion system and saving production costs.

FIG. 3a is a schematic view of the infusion mechanism module 110 according to the embodiment of the present invention. FIG. 3b is a side view of the assembly of the control mechanism module 100 and the infusion mechanism module 110 according to the embodiment of the present invention. FIG. 3c is a schematic top view of the lower case of the infusion mechanism module according to an embodiment of the present invention. FIG. 3d is a schematic top view of the lower case of the infusion mechanism module according to another embodiment of the present invention. FIG. 3e is a schematic view of the infusion mechanism module's infusion needle unit according to an embodiment of the present invention. FIG. 3f is a schematic view of the reservoir and cavity of the infusion mechanism module according to an embodiment of the present invention.

The skin patch drug infusion system further includes an infusion mechanism module 110 with a case. A mechanical unit, an electric control unit, and other auxiliary units for completing the drug infusion process are provided inside the case, which will be described in detail below. The case of the infusion mechanism module 110 may include multiple parts. As in the embodiment of the present invention, the case of the infusion system includes an upper case 111a and a lower case 111b.

As mentioned above, in the embodiment of the present invention, the infusion mechanism module 110 is provided with the second engaging portions 112, which is used to engaged and fasten the corresponding first engaging portions 102. The positions where the first engaging portions 102 and the second engaging portions 112 are provided correspondingly.

In the embodiment of the present invention, the infusion mechanism module 110 is provided with a second electrical connection 113. The second electrical connection 113 is electrically connected to the first electrical connection 103, thereby electrically connecting the control mechanism module 100 to the infusion mechanism module 100. Preferably, the second electrical connection 113 is multiple second electrical contacts 113. The technical advantages of the electrical contacts apply to both the first electrical contacts 103 on the control mechanism module 100 and the second electrical contacts 113 on the infusion mechanism module 110, which will not be described respectively in detail here. The second electrical contacts 113 are used to press against the corresponding first electrical contacts 103 to create an electrical connection between the control mechanism module 100 and the infusion mechanism module 110. The mutual pressing between these two corresponding electrical contacts disposed on different mechanism modules can improve the reliability of the electrical connection. Similar to first electrical contacts 103, one of the second electrical contact 113 includes a rigid metal pin and an elastic conductive member. Preferably, in the embodiment of the present invention, the second electrical contact 113 is a conductive spring.

Similarly, the conductive spring can improve the electrical connection performance. A groove is also arranged around the area where the second electrical contact 113 is disposed of, and a sealing member 115 is arranged in the groove. Similarly, the elasticity of the conductive spring can further improve the electrical connection performance.

Preferably, in the embodiment of the present invention, the two ends of the conductive spring have different diameters. And the diameter of the end exposed to the outside of the infusion mechanism module 110 is shorter than that of the end inside the infusion mechanism module 110. In this way, the conductive spring can be held in the case because of the longer diameter; Thus, when the control mechanism module 100 is not installed on the infusion mechanism module 110, the longer diameter of the inner end can prevent the conductive spring from detaching from the infusion mechanism module 110.

The embodiment of the present invention does not limit the position where second electrical contacts 113 are arranged, as long as it can be electrically connected to the corresponding first electrical contacts 103. Preferably, in the embodiment of the present invention, the upper case 111a of the infusion mechanism module 110 includes a convex portion 114 where the second electrical contacts 113 are disposed, as shown in FIG. 3a. The shape of the convex portion 114 corresponds to that of the concave 104 disposed on the control mechanism module 100, allowing the two portions to tightly fit each other and press the first electrical contacts 103 and the corresponding second electrical contacts 113 against each other to realize the electrical connection.

In other embodiments of the present invention, the convex portion 114 may be provided on the lower case 111b. When the infusion mechanism module 110 includes an integral case, the convex portion 114 will be a part of the integral case not specifically limited herein.

The method of assembling the control mechanism module 100 and the infusion mechanism module 110 to each other includes pressing the control mechanism module 100 on the infusion mechanism module 110 along with the thickness direction of the infusion mechanism module 110, thereby engaging the first engaging portion 102 and the second engaging portion 112, or pressing the control mechanism module 100 on the infusion mechanism module 110 along with the length direction of the infusion mechanism module 110. Alternatively, the control mechanism module 100 can be pressed along with any angle between the thickness direction and the length direction of the infusion mechanism module 110, making the first engaging portion 102 and the second engaging portion 112 engaged with each other. Preferably, in the implementation of the present invention, the method by which the control mechanism module 100 and the infusion mechanism module 110 are assembled is to press the control mechanism module 100 on the infusion mechanism module 110 along with the thickness direction of the infusion mechanism module 110, making the first engaging portion 102 and the second engaging portion 112 engaged with each other, as shown the installation direction in FIG. 3b.

In the embodiment of the present invention, the lower case 111b of the infusion mechanism module 110 further includes an outward extending portion 116. A block 117 is provided outside the outward extending portion 116, as shown in FIG. 3a. As mentioned above, the control mechanism module 100 is pressed to the engaging position along the thickness direction of the infusion mechanism module 110; thus, block 117 can prevent the control mechanism module 100 from detaching along the length direction of the infusion mechanism module 110, ensuring the normal operation of the infusion system. Obviously, in another embodiment of the present invention, if the control mechanism module 100 is pressed to the engaging position along with other directions, the control mechanism module 100 can also be prevented from detaching from the infusion mechanism module 110 by adjusting the position of the block 117.

It should be noted here that “outer” and “outside” are relative to the main body of the infusion mechanism module 110 and that they belong to a concept of relative position, as shown in FIG. 3a or FIG. 3b. The “outside” below have the same meaning as here.

In the embodiment of the present invention, the outer end of the outward extending portion 116 is also provided with a pressing portion 118 for releasing the blocking effect of block 117. While the user is replacing the infusion mechanism module 110, a finger presses the pressing portion 118, releasing the control mechanism module 100 from block 117. Then, the user can remove the control mechanism module 100 from the infusion mechanism module 110 with another two fingers.

Another embodiment of the present invention can also be provided with an unlocking hole 119 disposed of in the inner side of block 117. While the pressing portion 118 is pressed, a finger can enter the unlocking hole 119, thereby pushing the control mechanism module 100 out to separate the control mechanism module 100 from the infusion mechanism module 110. In the embodiment of the present invention, the unlocking hole 119 is square. The square unlocking hole 119 can facilitate the smooth entry of fingers. In other embodiments of the present invention, the unlocking hole 119 may also have other shapes, which is not specifically limited here.

The lower case 111b of the infusion mechanism module 110 is also provided with one or more crease grooves 140. Two crease grooves 140 are provided on both sides of the unlocking hole 119, as shown in FIG. 3c and FIG. 3d. After the crease groove, 140 is provided, the thickness or width of the lower case 111b at the crease groove 140 (as shown by the arrows in FIG. 3c and FIG. 3d) is reduced. When the user presses the pressing portion 118, the lower case 111b is easily broken at the crease groove 140, and the blocking of the control mechanism module 100 by block 117 is more smoothly released.

Preferably, in the embodiment of the present invention, two crease grooves 140 are provided at the two ends of block 117, respectively, as shown in FIG. 3c. In another embodiment of the present invention, the crease groove 140 is provided on two corresponding lateral sides of the unlocking hole 119, as shown in FIG. 3d.

The skin patch drug infusion system further includes a needle unit 121, used for infusing the drug to the subcutaneous tissue. As shown in FIG. 3e, needle unit 121 includes an infusion needle 1211 and a needle holder 1212. The infusion needle 1211 is fixedly placed on the needle holder 1212. The infusion needle 1211 includes a front end 1211a and a rear end 1211b, extending out of the needle holder 1212. The front end 1211a is used to communicate with the opening 20 of the drug storage unit 1110, while the rear end 1211b is inserted under the patient's skin.

As shown in FIG. 3f, the infusion mechanism module 100 is further provided with a cavity 30, including a first outlet 31 and a second outlet, that is, drug outlet 132b (as shown in FIG. 4b). The first outlet 31 is in sealed communication with the opening 20. Here, the sealed communication means that cavity 30 and the drug storage unit 131 communicate through opening 20 and the first outlet 31 without drug leaking. The second outlet, 132b, is sealed by an elastic seal 40. When the front end 1211a pierces the elastic seal 40, the infusion needle 1211, the cavity 30, the opening 20 and the drug storage unit 131 are in communication. Therefore, the drug can enter the infusion needle 1211 from the drug storage unit 131 to the rear end 1211b or be infused under the skin of the patient.

In the embodiment of the present invention, according to the actual needs of the user or patient, the volume of reservoir 131 is 1 mL-5 mL, preferably 1 mL-2 mL. If the volume of reservoir 131 is too large, the volume of the entire infusion mechanism module 110 will increase, thus affecting the user experience; if the volume is too small, the user's requirements cannot be met, and the infusion mechanism module 110 needs to be changed frequently, which will also affect the user experience.

An adhesive patch 150 is also provided on the bottom of the lower case 111b to attach the infusion system to the user's skin surface.

The skin patch drug infusion system further includes a filling module 120. In the embodiment of the present invention, the filling module 120 is a syringe 120 for inhaling insulin from a vial, such as an insulin vial, and then inserting it into the drug inlet 132a (as shown in FIG. 3a) to draw out the air in the fluid path and aspirate it, and finally fill the drug into the reservoir 131. As shown in FIG. 3f, the infusion mechanism module 110 is also provided with a third outlet: a drug inlet 132a, sealed by an elastic seal 50. In the embodiment of the present invention, the elastic seals 40 and 50 can be the same or different. There are no specific restrictions here, as long as they can be used for sealing. During the drug filling process, the needle of the syringe 120 will pierce through the elastic seal 50. The syringe 120, the cavity 30, the opening 20 and the reservoir 131 become four interconnecting parts. The air in the fluid path is drained into the syringe 120 by the negative pressure, which is generated by pulling back the push-pull rod of the syringe. After the air in the syringe is removed, the drug can also be filled into reservoir 131 from the syringe 120. Pull out syringe 120 after aspirating the air in the fluid path or transferring the drug into reservoir 131. Medicine inlet 132a will be automatically sealed due to the elastic seal 50. The elastic seal 50 helps to avoid drug leakage from reservoir 131 and prevent air from entering.

In the embodiment of the present invention, the volume of the filling module 120 is deliberately designed to be greater than the volume of the drug to be infused. After the filling module 120 withdraws a predetermined amount of drug, there will still be enough space for negative pressure to be generated to draw out the air in the fluid path into the filling module 120 and then aspirated, and finally fill the drug into the reservoir 131.

Preferably, the volume of the filling module 120 is deliberately designed to be greater than the volume of the reservoir 131, the maximum amount of drug to be infused can be filled into the reservoir during the drug filling and air aspirating process, which increases the use time of the drug, reduces the frequency of replacement of the drug infusion system, improves the user experience, and improves the accuracy of the actual drug volume filled into the reservoir, thereby improving the accuracy of the drug infusion.

Preferably, in the embodiment of the present invention, the volume of the filling module 120 is ImL-2 mL larger than the volume of the reservoir 131. For example, when the volume of the reservoir 131 is 1 mL, the volume of the filling module 120 will be 2 mL-3 mL. When the volume of the reservoir 131 is 2 mL, the volume of the filling module 120 will be 3 mL-4 mL, and when the volume of the reservoir 131 is 5 mL, the volume of the filling module 120 will be 6 mL-7 mL. The specific volume of the filling module 120 and the reservoir 131 is not specifically limited here. In another embodiment of the present invention, the volume of the filling module 120 is at least 20% larger than the volume of the reservoir 131, and it can be 30%, 50%, or 100%, which is not specifically limited here.

FIG. 4a and FIG. 4b are two schematic views of the internal mechanism module 130 of the infusion mechanism module 110 of the embodiment of the present invention from two perspectives, respectively.

In the embodiment of the present invention, the internal mechanism module 130 includes mechanical units and electronic control units used to realize the infusion function, such as a drug reservoir 131, a drug inlet 132a, a drug outlet 132b, a power supply 133, a driving wheel 134, a screw 135, a circuit board (not shown), a driving unit (not shown), etc. The movement of the driving unit drives the driving wheel 134 to rotate, thus making the screw 135 push the piston (not shown) in the drug reservoir 131 forward, realizing the drug infusion.

In the embodiment of the present invention, the power supply 133 is a conventional button battery. In other embodiments of the present invention, the power supply 133 may also be other types of batteries, as long as it can meet the requirements for supplying power to the infusion system. Preferably, in the embodiment of this present invention, the type of the power supply 133 is a double-row battery pack; that is, two rows of button batteries are respectively arranged on both sides of the driving wheel 134, as shown in FIG. 4b. Conventionally, the discharge capacity of button batteries is low. The double-row button battery pack can reduce the discharge level of each battery, thereby extending the service life of the battery. Furthermore, the double-row design of the power supply 133 can make full use of the internal space and improve the integration of the internal mechanism module in the infusion system.

The infusion mechanism module 110 in the embodiment of the present invention is also provided with a circuit board or multiple three-dimensional circuits coated on the surface of a part of the mechanism module for supplying power to specific structural units. The circuit board is a hard/rigid circuit board or a flexible circuit board. Preferably, in the embodiment of the present invention, the circuit board is flexible. The shape of the flexible circuit board is plastic, allowing it to be flexibly designed according to the internal space of the infusion mechanism module 110. At the same time, multiple connection ends can be provided on the flexible circuit board to be electrically connected to second electrical contacts 113, thereby connecting the circuits of the control mechanism module 100 and the infusion mechanism module 110, allowing the infusion system perform drug infusion function.

An elastic conductor 136 is also provided inside the infusion mechanism module 130, as shown in FIG. 4a. The elastic conductor 136 is electrically connected to the power supply 133, and the specific connection end on the circuit board (or three-dimensional circuit), thereby supplying power to specific structural units.

Similar to the elastic conductive member above mentioned, the type of the elastic conductor 136 includes a conductive spring, a conductive leaf spring, a conductive rubber, a conductive silica gel, etc., which are not specifically limited herein, as long as they can meet the requirements for electrically connecting the power supply 133 to specific connection ends on the circuit board (or three-dimensional circuit). Preferably, in the embodiment of the present invention, the elastic conductor 136 is the conductive leaf spring. Since the infusion mechanism module 110 has a double-row battery pack, the multiple conductive leaf springs are also designed as a double-row pack, as shown in FIG. 4a.

The elastic conductor 136 can realize a direct electrical connection between the power supply 133 and the specific structural units, which helps to optimize the internal circuit design and reduce the complexity of the internal mechanism module.

FIG. 5 is a flow chart of the drug filling process, according to an embodiment of the present invention.

In the embodiment of the present invention, the drug filling method applied to the patch type drug infusion system is mainly used to withdraw the drug, such as insulin, from the vial to the reservoir 131 of the infusion mechanism module 110. The filling method can include:
Step S410: Withdraw the drug to be infused into the filling module, and the volume of the filling module 120 is deliberately designed to be greater than the volume of the drug to be infused;
Step S420: Insert the filling module 120 into the drug inlet 132a of the infusion mechanism module 110 and draw out the air in the fluid path of the infusion mechanism module 110;
Step S430: Pull out the filling module 120 and aspirate the air;
Step S440: Insert the filling module 120 into the drug inlet 132a of the infusion mechanism module 110 again and fill it into reservoir 131 of the infusion mechanism module 110.

In this way, the volume of the filling module 120 is deliberately designed to be greater than the volume of the drug to be infused; The excess space in the filling module generates negative pressure to draw out the air in the fluid path, reducing the risk of air being infused into the body, mitigating safety hazards, and improving user experience.

Specifically, in the embodiment of the present invention, the filling module 120 is a syringe 120. In step S410, the push-pull rod of the syringe 120 is pulled back to extract a certain amount of air into the syringe 120 and then fill it into the vial so that the positive pressure in the vial compelled the drug into the syringe 120. Since the drug volume to be infused is always deliberately designed to be smaller than the filling module 120 volume, the syringe 120 will never be full filled with the drug, and there will always be extra space in the syringe 120.

In step S420, the syringe needle is vertically inserted into the drug inlet 132a of the infusion mechanism module 110. As mentioned above, after the needle of the syringe 120 pierces the elastic sealing 50 and is inserted into the drug inlet 132a, the syringe 120, the cavity 30, the opening 20 and the reservoir 131 are communicated. Pull the push-pull rod of the syringe 120 to the top position of the syringe 120. Since there is still space in the syringe 120 that has not been filled with drugs, pulling the push-pull rod of the syringe 120 can create negative pressure to draw out the air in the fluid path, so air bubbles are gathered and floating up into the syringe 120. Loosen the push-pull rod, the push-pull rod will automatically return its initial position, but the air bubbles will not be pushed into reservoir 131 due to the air pressure.

In the embodiment of the present invention, an air aspirating step may be included between steps S410 and S420. Specifically, flick the syringe 120 to make the air bubbles float to the top of the syringe 120, and slowly push the push-pull rod to aspirate air. This air aspirating process can be performed in the vial or after the syringe 120 is removed from the vial, which is not limited here.

In step S430, pull out the needle of the syringe 120 from the drug inlet 132a of the infusion mechanism module 110. At this time, due to the existence of the elastic sealing 50, after the needle is pulled out, the drug inlet 132a will be automatically sealed. Flick the syringe 120 again to make the air bubbles float to the top of the syringe 120, and slowly push the push-pull rod to aspirate the air.

In step S440, insert the filling module 120 into the drug inlet 132a of the infusion mechanism module 110 again and slowly push the push-pull rod to fill the drug into the reservoir 131, pull out the syringe 120. The drug inlet 132a will be automatically sealed by the elastic seal 50, and the drug filling process is completed.

In the embodiment of the present invention, the volume of the filling module 120 is deliberately designed to be greater than the volume of the drug to be infused. After the filling module 120 inhales a predetermined amount of drug, there is still enough space for generating negative pressure to draw out the air in the fluid path into the filling module 120 and aspirate the air, then fill the drug into the reservoir 131.

In the embodiment of the present invention, the volume of the filling module 120 is deliberately designed to be greater than the volume of the reservoir 131, after the filling module 120 withdraws a predetermined amount of drug, such as the maximum volume of the reservoir 131, there is still enough space for generating negative pressure to draw out the air in the fluid path into the filling module 120 and aspirate the air, then fill the drug into the reservoir 131. This increases the drug's use time, reduces the frequency of replacement of the drug infusion system, improves the user experience, and improves the accuracy of the actual drug volume filled into the reservoir, thereby improving the accuracy of the drug infusion.

Preferably, in the embodiment of the present invention, the volume of the filling module 120 is ImL-2 mL larger than the volume of the reservoir 131. For example, when the volume of the reservoir 131 is 1 mL, the volume of the filling module 120 will be 2 mL-3 mL. When the volume of the reservoir 131 is 2 mL, the volume of the filling module 120 will be 3 mL-4 mL, and when the volume of the reservoir 131 is 5 mL, the volume of the filling module 120 will be 6 mL-7 mL. The specific volume of the filling module 120 and the reservoir 131 is not specifically limited here. In another embodiment of the present invention, the volume of the filling module 120 is at least 20% larger than the volume of the reservoir 131, and it can be 30%, 50%, or 100%, which is not specifically limited here.

In the embodiment of the present invention, the drug infusion system's drug filling process can be performed before or after module 110 and control mechanism 100 are electrically connected, which is not limited here. Users can operate it according to personal habits and preferences and improve the diversity and convenience of operations.

FIG. 6 is a flow chart of the drug filling process according to another embodiment of the present invention.

In the embodiment of the present invention, the drug filling method applied to the patch type drug infusion system is mainly used to withdraw the drug, such as insulin, from the vial and fill it into reservoir 131 of the infusion mechanism module 110. The filling method can comprise:
Step S510: Withdraw the drug to be infused from a vial into the filling module 120;
Step S520: Insert the filling module 120 into the drug inlet 132a of the infusion mechanism module and fill the drug into the reservoir;
Step S530: Pull the push-pull rod of the filling module 120 and draw out the air in the fluid path of the infusion mechanism module 110.

In this way, after the drug was filled into reservoir 131, the filling module 120 is used to draw out and aspirate the air in the fluid path, reducing the risk of air being infused into the body, lowering safety hazards and improve user experience.

Specifically, in the embodiment of the present invention, the filling module 120 is a syringe 120. In step S510, the push-pull rod of the syringe 120 is pulled to extract a certain amount of air into the syringe 120 and then fill it into the reservoir 131 so that the positive pressure in the vial makes the drug to be infused compelled into the syringe 120.

In step S520, the syringe needle is vertically inserted into the drug inlet 132a of the infusion mechanism module 110, and push the push-pull rod slowly to fill the drug into reservoir 131.

In the embodiment of the present invention, an air aspirate step may be included between steps S510 and S520. Specifically, flick the syringe 120 to make the air bubbles float to the top of the syringe 120, and slowly push the push-pull rod to aspirate air. This air aspirating process can be performed in the vial or after the syringe 120 is removed from the vial, which is not limited here.

In step S530, pull the push-pull rod of the filling module 120, draw out the air in the fluid path of the infusion mechanism module 110, and pull out the filling module 120. The air in the fluid path is therefore aspirated, reducing the risk of air being infused into the body, lowering safety hazards, and improving user experience.

In the embodiment of the present invention, the drug infusion system's drug filling process can be performed before or after module 110 and control mechanism 100 are electrically connected, which is not limited here. Users can operate it according to personal habits and preferences and improve the diversity and convenience of operations.

In summary, the present invention discloses a skin patch drug infusion system and a drug filling method applied to a skin patch drug infusion system. The volume of the filling module is deliberately designed to be greater than the volume of the drug to be infused. The excess space in the filling module generates negative pressure to draw out the air in the fluid path, reducing the risk of air being infused into the body, lowering safety hazards, and improving user experience. The present invention also discloses another drug filling method applied to a skin patch drug infusion system. After the drug was filled into the reservoir, the filling module draws the air out and aspirates the air in the fluid path, reducing the risk of air infusing into the body, lowering safety hazards, and improving user experience.

While the invention has been described in detail regarding the specific embodiments of the present invention, it should be understood that it will be appreciated by those skilled in the art that the above embodiments may be modified without departing from the scope and spirit of the invention. The appended claims define the scope of the invention.

1. A skin patch drug infusion system, comprising:
an infusion mechanism module, wherein the infusion mechanism includes:
a reservoir, for accommodating a drug to be infused, and provided with a drug inlet and a drug outlet;
an infusion needle, wherein one end of the infusion needle is communicated with the drug outlet of the reservoir, and an other end of the infusion needle is inserted subcutaneously implanting drug infusion;
a control mechanism module, worked collaboratively with the infusion mechanism module to regulate drug infusion;
an adhesive patch, for attaching the infusion mechanism module and/or the control mechanism module to a skin surface; and
a filling module, for filling the drug to be infused into the reservoir via the drug inlet, wherein a volume of the filling module is deliberately designed to be greater than a volume of the drug to be infused, and an excess space in the filling module is used to generate negative pressure to extract air in a fluid path.

2. A skin patch drug infusion system of claim 1, wherein
the volume of the filling module is deliberately designed to be greater than a volume of the reservoir.

3. A skin patch drug infusion system of claim 2, wherein
the volume of the filling module is 1 mL-2 mL greater than the volume of the reservoir.

4. A skin patch drug infusion system of claim 3, wherein
the reservoir volume is 1 mL-5 mL.

5. A skin patch drug infusion system of claim 4, wherein
the volume of the reservoir is 1 mL-2 mL.

6. A skin patch drug infusion system of claim 2, wherein
the volume of the filling module is at least 20% greater than the volume of the reservoir.

7. A skin patch drug infusion system of claim 1,
further including an elastic seal at the drug inlet, which automatically seals the drug inlet to prevent the drug from leaking after the drug is filled into the reservoir.

8. A skin patch drug infusion system of claim 1, wherein
the infusion mechanism module and the control mechanism module are detachable to each other, and the control mechanism module is reusable.

9. A skin patch drug infusion system of claim 1, wherein
the infusion mechanism module and the control mechanism module are disposed in one housing, discarded together after a single-use.

10. (canceled)

11. (canceled)

12. (canceled)

13. (canceled)

14. (canceled)

15. A drug filling method of skin patch drug infusion system of claim 1 for filling the drug to be infused into the infusion mechanism module, comprising:
Step 1: Withdrawing the drug to be infused from a vial into the filling module, wherein the volume of the filling module is deliberately designed to be greater than the volume of the drug to be infused;
Step 2: Inserting the filling module into the drug inlet of the infusion mechanism module and draw the air in the fluid path of the infusion mechanism module into the filling module;
Step 3: Pulling out the filling module and aspirate the air; and
Step 4: Inserting the filling module into the drug inlet of the infusion mechanism module again and filling it into the reservoir of the infusion mechanism module.

16. A drug filling method of claim 15,
further including air aspirating between step 1 and step 2.

17. A drug filling method of claim 16, wherein
the air aspirating is performed in the vial or after the filling module being pulled out from the vial.

18. A drug filling method of claim 15, wherein
the volume of the filling module is deliberately designed to be greater than a volume of the reservoir.

19. A drug filling method of claim 18, wherein
the drug filling method is performed after or before the infusion mechanism module and control mechanism module electrically connected.

20. A drug filling method of skin patch drug infusion system of claim 1 for filling the drug to be infused into the infusion mechanism module, comprising:
Step 1: Withdrawing the drug to be infused from a vial into the filling module;
Step 2: Inserting the filling module into the drug inlet of the infusion mechanism module and fill the drug into the reservoir;
Step 3: Pulling a push-pull rod of the filling module and draw out the air in the fluid path of the infusion mechanism module.

21. A drug filling method of claim 20,
further including air aspirating between step 1 and step 2.

22. A drug filling method of claim 21, wherein
the air aspirating is performed in the vial or after the filling module being pulled out from the vial.

23. A drug filling method of claim 20, wherein
the drug filling method is performed before or after the infusion mechanism module and control mechanism module are electrically connected.
